SEQUENCE LISTING (4 sequences)

SEQ ID NO: 1 - protein, 183 aa, Homo sapiens
MTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 2 - DNA, 591 bp, Homo sapiens
atggctgatatcggatccatacatatgacgaccgcgtccacctcgcaggtgcgccagaactaccaccaggactcagaggccgccatcaaccgccagatcaacctggagctctacgcctcctacgtttacctgtccatgtcttactactttgaccgcgatgatgtggctttgaagaactttgccaaatactttcttcaccaatctcatgaggagagggaacatgctgagaaactgatgaagctgcagaaccaacgaggtggccgaatcttccttcaggatatcaagaaaccagactgtgatgactgggagagcgggctgaatgcaatggagtgtgcattacatttggaaaaaaatgtgaatcagtcactactggaactgcacaaactggccactgacaaaaatgacccccatttgtgtgacttcattgagacacattacctgaatgagcaggtgaaagccatcaaagaattgggtgaccacgtgaccaacttgcgcaagatgggagcgcccgaatctggcttggcggaatatctctttgacaagcacaccctgggagacagtgataatgaaagctaacctaggcacctcgag

SEQ ID NO: 3 - DNA, 3216 bp, Homo sapiens
ttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcgacggattctagaactagtggatcccccatcacaagtttgtacaaaaaagcaggctttaaaggaaccaattcagtcgactggatccatacatatgacgaccgcgtccacctcgcaggtgcgccagaactaccaccaggactcagaggccgccatcaaccgccagatcaacctggagctctacgcctcctacgtttacctgtccatgtcttactactttgaccgcgatgatgtggctttgaagaactttgccaaatactttcttcaccaatctcatgaggagagggaacatgctgagaaactgatgaagctgcagaaccaacgaggtggccgaatcttccttcaggatatcaagaaaccagactgtgatgactgggagagcgggctgaatgcaatggagtgtgcattacatttggaaaaaaatgtgaatcagtcactactggaactgcacaaactggccactgacaaaaatgacccccatttgtgtgacttcattgagacacattacctgaatgagcaggtgaaagccatcaaagaattgggtgaccacgtgaccaacttgcgcaagatgggagcgcccgaatctggcttggcggaatatctctttgacaagcacaccctgggagacagtgataatgaaagctaacctaggcacctcgagatatctagacccagctttcttgtacaaagtggtgatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccgcataggccactagtggatctgatatcacctaataacttcgtatagcatacattatacgaagttatattaagggttctcgagagctcgttttatttaggttctatcgaggagaaaaagcgacaagaagagatagaccatggataaatgattatgttctaaacactcctcagaagctcatcgaactgtcatcctgcgtgaagattaaaatccaacttagaaatttcgagcttacggagacaatcatatgggagaagcaattggaagatagaaaaaaggtactcggtacataaatatatgtgattctgggtagaagatcggtctgcattggatggtggtaacgcatttttttacacacattacttgcctcgagcatcaaatggtggttattcgtggatctatatcacgtgatttgcttaagaattgtcgttcatggtgacacttttagctttgacatgattaagctcatctcaattgatgttatctaaagtcatttcaactatctaagatgtggttgtgattgggccattttgtgaaagccagtacgccagcgtcaatacactcccgtcaattagttgcaccatgtccacaaaatcatataccagtagagctgagactcatgcaagtccggttgcatcgaaacttttacgtttaatggatgaaaagaagaccaatttgtgtgcttctcttgacgttcgttcgactgatgagctattgaaacttgttgaaacgttgggtccatacatttgccttttgaaaacacacgttgatatcttggatgatttcagttatgagggtactgtcgttccattgaaagcattggcagagaaatacaagttcttgatatttgaggacagaaaattcgccgatatcggtaacacagtcaaattacaatatacatcgggcgtttaccgtatcgcagaatggtctgatatcaccaacgcccacggggttactggtgctggtattgttgctggcttgaaacaaggtgcgcaagaggtcaccaaagaaccaaggggattattgatgcttgctgaattgtcttccaagggttctctagcacacggtgaatatactaagggtaccgttgatattgcaaagagtgataaagatttcgttattgggttcattgctcagaacgatatgggaggaagagaagaagggtttgattggctaatcatgaccccaggtgtaggtttagacgacaaaggcgatgcattgggtcagcagtacagaaccgtcgacgaagttgtaagtggtggatcagatatcatcattgttggcagaggacttttcgccaagggtagagatcctaaggttgaaggtgaaagatacagaaatgctggatgggaagcgtaccaaaagagaatcagcgctccccattaattatacaggaaacttaatagaacaaatcacatatttaatctaatagccacctgcattggcacggtgcaacactacttcaacttcatcttacaaaaagatcacgtgatctgttgtattgggatctctagacctaataacttcgtatagcatacattatacgaagttatattaagggttgtcgacctg

SEQ ID NO: 4 - DNA, 4379 bp, Homo sapiens
cggtcaagaagacatatttgacctcttaacaggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcgacggattctagaactagtggatcccccatcacaagtttgtacaaaaaagcaggctttaaaggaaccaattcagtcgactggatccatacatatgacgaccgcgtccacctcgcaggtgcgccagaactaccaccaggactcagaggccgccatcaaccgccagatcaacctggagctctacgcctcctacgtttacctgtccatgtcttactactttgaccgcgatgatgtggctttgaagaactttgccaaatactttcttcaccaatctcatgaggagagggaacatgctgagaaactgatgaagctgcagaaccaacgaggtggccgaatcttccttcaggatatcaagaaaccagactgtgatgactgggagagcgggctgaatgcaatggagtgtgcattacatttggaaaaaaatgtgaatcagtcactactggaactgcacaaactggccactgacaaaaatgacccccatttgtgtgacttcattgagacacattacctgaatgagcaggtgaaagccatcaaagaattgggtgaccacgtgaccaacttgcgcaagatgggagcgcccgaatctggcttggcggaatatctctttgacaagcacaccctgggagacagtgataatgaaagctaacctaggcacctcgagatatctagacccagctttcttgtacaaagtggtgatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccgcataggccactagtggatctgatatcacctaataacttcgtatagcatacattatacgaagttatattaagggttctcgagagctcgttttatttaggttctatcgaggagaaaaagcgacaagaagagatagaccatggataaatgattatgttctaaacactcctcagaagctcatcgaactgtcatcctgcgtgaagattaaaatccaacttagaaatttcgagcttacggagacaatcatatgggagaagcaattggaagatagaaaaaaggtactcggtacataaatatatgtgattctgggtagaagatcggtctgcattggatggtggtaacgcatttttttacacacattacttgcctcgagcatcaaatggtggttattcgtggatctatatcacgtgatttgcttaagaattgtcgttcatggtgacacttttagctttgacatgattaagctcatctcaattgatgttatctaaagtcatttcaactatctaagatgtggttgtgattgggccattttgtgaaagccagtacgccagcgtcaatacactcccgtcaattagttgcaccatgtccacaaaatcatataccagtagagctgagactcatgcaagtccggttgcatcgaaacttttacgtttaatggatgaaaagaagaccaatttgtgtgcttctcttgacgttcgttcgactgatgagctattgaaacttgttgaaacgttgggtccatacatttgccttttgaaaacacacgttgatatcttggatgatttcagttatgagggtactgtcgttccattgaaagcattggcagagaaatacaagttcttgatatttgaggacagaaaattcgccgatatcggtaacacagtcaaattacaatatacatcgggcgtttaccgtatcgcagaatggtctgatatcaccaacgcccacggggttactggtgctggtattgttgctggcttgaaacaaggtgcgcaagaggtcaccaaagaaccaaggggattattgatgcttgctgaattgtcttccaagggttctctagcacacggtgaatatactaagggtaccgttgatattgcaaagagtgataaagatttcgttattgggttcattgctcagaacgatatgggaggaagagaagaagggtttgattggctaatcatgaccccaggtgtaggtttagacgacaaaggcgatgcattgggtcagcagtacagaaccgtcgacgaagttgtaagtggtggatcagatatcatcattgttggcagaggacttttcgccaagggtagagatcctaaggttgaaggtgaaagatacagaaatgctggatgggaagcgtaccaaaagagaatcagcgctccccattaattatacaggaaacttaatagaacaaatcacatatttaatctaatagccacctgcattggcacggtgcaacactacttcaacttcatcttacaaaaagatcacgtgatctgttgtattgggatctctagacctaataacttcgtatagcatacattatacgaagttatattaagggttgtcgacctgtcagatatagtcggattgcccttttaagcaatcaatagtgaaggtcgatattgggaactgtggtggttggcattcgagtttatcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatggttagagttgctattaacggtttcggtagaatcggtagattggtcatgagaattgctttgtctagaccaaacgtcgaagttgttgctttgaacgacccattcatcaccaacgactacgctgcttacatgttcaagtacgactccactcacggtagatacgctggtgaagtttcccacgatgacaagcaca

(19) United States
(12) Patent Application Publication
(10) Pub. No.: US 2024/0252568 A1
(43) Pub. Date: Aug. 1, 2024

(54) RECOMBINANT HOSTS COMPRISING FERRITIN OR HOMOLOGUES THEREOF

(71) Applicant: Sidero Bioscience, LLC, Hummelstown, PA (US)

(72) Inventors: Ralph Keil, Palmyra, PA (US); James Connor, Hershey, PA (US); Darren Wolfe, Cincinnati, OH (US)

(21) Appl. No.: 18/561,439

(22) PCT Filed: May 19, 2022
(86) PCT No.: PCT/US2022/030118

§ 371 Date: Nov. 16, 2023

Related U.S. Application Data
(60) Provisional application No. 63/190,257, filed on May 19, 2021.

(51) Int. Cl. A61K 36/064; A23L 33/14; A61K 33/26; A61K 38/00; C07K 14/47; C12N 1/16; C12R 1/865

(52) U.S. Cl. CPC A61K 36/064; A23L 33/14; A61K 33/26; C07K 14/47; C12N 1/16; A61K 38/00; C12R 2001/865

(57) ABSTRACT

Disclosed herein are recombinant hosts comprising at least a first set of chromosomes and a second set of chromosomes, wherein at least one of the first set of chromosomes and the second set of chromosomes comprises a recombinant gene encoding ferritin or a homologue thereof. Also disclosed herein are recombinant hosts comprising a prototroph comprising a recombinant gene encoding ferritin or a homologue thereof. The recombinant hosts may be included in compositions, ingestible items, dietary supplements or pharmaceutical compositions. Also disclosed herein are methods of administering to a subject a composition of the present invention.

### FIELD

The present disclosure relates to recombinant hosts comprising ferritin or homologues thereof.

### BACKGROUND

Deficiencies in iron absorption or excesses in iron loss lead to non-optimal blood and/or tissue iron levels with a wide variety of symptoms. The current industry standard for iron replacement therapy is oral administration of iron sulfate supplements. However, such supplements have limited efficacy because the iron is poorly absorbed or the supplement is dependent on environmental conditions to generate a consistent level of iron. Use of iron sulfate supplements can also lead to gastrointestinal discomfort.

### SUMMARY

Disclosed herein is a recombinant host comprising: at least a first set of chromosomes and a second set of chromosomes, wherein at least one of the first set of chromosomes and the second set of chromosomes comprises a recombinant gene encoding ferritin or a homologue thereof.

Also disclosed herein is a recombinant host comprising: a prototroph comprising a recombinant gene encoding ferritin or a homologue thereof.

Also disclosed herein are compositions comprising a recombinant host of the present invention.

Also disclosed herein are ingestible items comprising a composition of the present invention.

Also disclosed herein are dietary supplements comprising a composition of the present invention.

Also disclosed herein are pharmaceutical compositions comprising a composition of the present invention.

Also disclosed herein are methods of administering to a subject a composition of the present invention.

### BRIEF DESCRIPTION OF THE DRAWINGS

FIG. 1 shows the amino acid sequence of human H-ferritin (SEQ ID NO:1).

FIG. 2 shows the nucleic acid sequence of human H-ferritin (SEQ ID NO:2).

FIG. 3 is a schematic showing the structure of a ferritin expression cassette containing the human H-ferritin gene, FTH1, and the selectable URA3 gene. The ferritin expression cassette was amplified by PCR from plasmid RLK/pL5659 using oligonucleotide primers 0-730 and 0-731. Double horizontal lines indicate 3.2 kbp ferritin expression cassette; open arrows indicate locations of primers with arrow indicating direction of DNA synthesis (5′ to 3′); hatched arrows indicate open-reading frames (ORFs) with point of arrow indicating 3′ end of genes (carboxy-terminus of the encoded protein); open arrow indicates the location of the constitutive, highly expressed yeast TDH3 promoter with the arrow showing the direction of transcription (5′ to 3′); open block indicates the yeast CYC1 terminator.

FIG. 4 shows the nucleic acid and amino acid sequences of the expression cassette shown in FIG. 3 (SEQ ID NO:3).

FIG. 5 is a schematic illustrating the chromosomal integration of a ferritin expression cassette containing the TDH3 promoter. Double horizontal lines indicate 3.2 kbp ferritin expression cassette; crosshatched arrows indicate locations of primers with arrow indicating direction of DNA synthesis (5′ to 3′); hatched arrows indicate open-reading frames (ORFs) with points of arrows indicating 3′ end of genes (carboxy-terminus of the encoded protein); open arrow indicates the location of the constitutive, highly expressed yeast TDH3 promoter with the arrow showing the direction of transcription (5′ to 3′); open block indicates the yeast CYC1 terminator.

FIG. 6 shows the nucleic acid sequence of the chromosomally integrated ferritin expression cassette at TDH3 (SEQ ID NO:4) shown in FIG. 5.

FIG. 7 compares the hemoglobin recovery in rats treated with a diploid yeast-ferritin complex compared to rats treated with a haploid yeast-ferritin complex in Example 1.

FIG. 8 shows the hematocrit recovery in rats treated with a diploid yeast-ferritin complex compared to rats treated with a haploid yeast-ferritin complex in Example 1.

FIG. 9A shows the iron content of a haploid yeast sample and a diploid yeast sample based on weight percent of a 1 g sample of yeast.

FIG. 9B shows the H-ferritin content of a haploid yeast sample and a diploid yeast sample.

### DETAILED DESCRIPTION

For purposes of the following detailed description, it is to be understood that the invention may assume various alternative variations and step sequences, except where expressly specified to the contrary. Moreover, other than in any operating examples, or where otherwise indicated, all numbers such as those expressing values, amounts, percentages, ranges, subranges, and fractions may be read as if prefaced by the word “about,” even if the term does not expressly appear. Accordingly, unless indicated to the contrary, the numerical parameters set forth in the following specification and attached claims are approximations that may vary depending upon the desired results to be obtained by the present invention. At the very least, and not as an attempt to limit the application of the doctrine of equivalents to the scope of the claims, each numerical parameter should at least be construed in light of the number of reported significant digits and by applying ordinary rounding techniques. Where a closed or open-ended numerical range is described herein, all numbers, values, amounts, percentages, subranges, and fractions within or encompassed by the numerical range are to be considered as being specifically included in and belonging to the original disclosure of this application as if these numbers, values, amounts, percentages, subranges, and fractions had been explicitly written out in their entirety.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

As used herein, unless indicated otherwise, a plural term can encompass its singular counterpart and vice versa, unless indicated otherwise. For example, although reference is made herein to “a” species of yeast or “a” set of chromosomes, a combination (i.e., a plurality) of these components can be used. In addition, in this application, the use of “or” means “and/or” unless specifically stated otherwise, even though “and/or” may be explicitly used in certain instances.

As used herein, “including.” “containing.” and like terms are understood in the context of this application to be synonymous with “comprising” and are therefore open-ended and do not exclude the presence of additional undescribed and/or unrecited elements, materials, ingredients and/or method steps.

As used herein, “consisting of” is understood in the context of this application to exclude the presence of any unspecified element, ingredient and/or method step.

As used herein, “consisting essentially of” is understood in the context of this application to include the specified elements, materials, ingredients and/or method steps “and those that do not materially affect the basic and novel characteristic(s)” of what is being described.

As used herein, “recombinant host” means a host, the genome of which is augmented or introduced by at least one recombinant gene. “Augmented” or “introduced” refers to augmentation or introduction caused by human intervention. Exemplary recombinant hosts include but are not limited to plants, fungi, such as yeast, bacteria, or animals, such as a canine, a porcine or a bovine.

As used herein, “recombinant gene” is a nucleic acid sequence in which one or more genes or segments of genes have been inserted, resulting in a new genetic combination. A recombinant gene is generally a gene that is not naturally found in the context in which it is used. A recombinant gene may be a gene that is integrated into the chromosome of a recombinant host at an integration site at which the sequence does not naturally occur. The recombinant gene can be a DNA sequence from a species other than the recombinant host. The recombinant gene can be one or more additional copies of a gene that naturally occurs in the recombinant host inserted into additional locations of the recombinant host's genome to allow for overexpression or altered expression of the gene product of that DNA sequence.

As used herein, “haploid” means a cell that contains one set of chromosomes.

As used herein, “one set of chromosomes” means the genome of a haploid.

As used herein, “diploid” means a cell that contains two homologous sets of chromosomes.

As used herein, “polyploid” means a cell that contains more than two homologous sets of chromosomes.

As used herein, “aneuploid” means a cell that contains an abnormal number of chromosomes. An “abnormal number of chromosomes” means a number of chromosomes that is different than from the usual number of chromosomes in a given organism. For example, an S. cerevisiae cell comprises 16 chromosomes. An aneuploid S. cerevisiae cell may comprise, as examples, 15 chromosomes or 17 chromosomes (i.e., any number that is not 16).

As used herein, “homologous” means pairs of chromosomes of similar centromere position, gene composition, and length.

As used herein, “two sets of chromosomes” means the genome of a diploid.

As used herein, “homologue” means one chromosome of a homologous pair.

As used herein, “prototroph” or “prototrophic” means a microorganism that can synthesize its nutrients from inorganic material.

As used herein, “mate”, “mated”, or “mates” is the process whereby two cells of opposite mating types, typically haploids, fuse, generally forming a diploid.

As used herein, “ferritin” is a globular protein that acts as a primary intracellular iron-storage protein in most organisms, including prokaryotes and eukaryotes. Ferritin is a large (nearly 480 kDa) multi-subunit complex comprising 24 polypeptide subunits and is capable of containing as many as 4.500 atoms of iron ions (Fe²⁺ or Fe³⁺) within a hydrous iron oxide core.

As used herein, “ingestible” means capable of being taken into the body orally.

As used herein, “medical food” means a food that is designed to be consumed or administered enterally for the treatment of a disease that has distinctive nutritional requirements that cannot be met solely by normal diet.

As used herein, “dietary supplement” or “nutritional supplement” means something that is consumed to supplement a subject's diet in addition to meals.

As used herein, “composition” means a solution, dispersion, or solid, such as a powder.

As used herein, “pharmaceutical composition” means any chemical or biological composition, material, agent, or the like that is capable of inducing a therapeutic or metabolic effect when properly administered to a subject, including the composition, material, agent or the like in an inactive form and active metabolites thereof, where such active metabolites may be formed in vivo.

As used herein, “subject” or “patient” means animals, including mammals, including humans, a canine, a feline, a bovine, an equine, a porcine, a primate, and/or a rodent.

As used herein, “administering” an amount (e.g., a dose) of a composition may be done by the subject himself/herself or another subject (e.g., a medical professional, a caretaker, a family member). The composition may be provided by the subject or the administrator for the subject along with instructions for the administration of the composition (e.g., written instructions on the label of a container containing the composition).

As used herein, “treat”, “treatment”, or “treating” means a therapeutic, prophylactic, or preventative measure provided to a patient or subject with the intention of preventing the development or altering the pathology or symptoms experienced by the patient or subject, such as, e.g., those resulting from a disorder, which may include an iron deficiency disorder or a gut microbiome disorder. A “treatment” administered to a patient or subject may achieve any clinical or quantitatively measurable reduction in the condition for which the patient or subject is being treated up to and including complete elimination. “Treatment” refers to both therapeutic treatment and prophylactic or preventative measures. “Treatment” may also be specified as palliative care. “Treatment” may also include administration of the composition as a dietary iron source. Those in need of treatment include those already with one or more iron-deficiency disorders, those in which the disorder is to be prevented, and those who may benefit from iron supplementation.

As used herein, “dietary management” means treatment of a condition through administration of a medical food, a food ingredient, or a dietary supplement.

As used herein, “salt” means an ionic compound made up of metal cations and non-metallic anions and having an overall electrical charge of zero. Salts may be hydrated or anhydrous.

As used herein, “dry matter,” with respect to a composition of the present invention, means that the composition has no more than 10% water by weight based on total weight of the composition.

As used herein, “dry matter basis” means a method of expressing the concentration of a component in a composition by expressing the component's concentration in terms of the dry matter content.

As used herein, “iron complexes” or “organic-iron complexes” are compounds which contain iron in the (II) or (III) oxidation state, complexed with or otherwise bound to (e.g., an ionic bond) an organic compound. Examples of suitable organic-iron complexes include, but are not limited to, iron polymer complexes, iron carbohydrate complexes, and iron aminoglycosan complexes.

As used herein, “closely linked” means a first gene is within five centimorgans of a second gene.

As used herein, “centimorgan” means a unit of measurement that is equal to a 1% chance that a first marker on a chromosome will become separated from a second marker on the same chromosome due to crossing over in a single generation.

As used herein, “iron” refers to elemental iron, which may be in the form of a salt, such as but not limited to iron oxide, iron sulfate, and iron hydride.

As used herein, “iron deficient” or “iron deficiency” refers to a condition in which subjects have a TSAT of ≤25% and a serum ferritin concentration of ≤100 ng/mL.

As used herein, “anemia” refers to a condition in which subjects have a hemoglobin concentration of less than 13 g/dL; subjects may exhibit symptoms commonly associated with disorders or diseases related to iron deficiency, iron uptake and/or iron metabolism based on responses to a health-related quality of life questionnaire. As used herein, “iron deficiency anemia” refers to a condition in which subjects have a TSAT of less than 20%, a serum ferritin concentration of less than 50 ng/mL, and a hemoglobin concentration of less than 13 g/dL. Subjects may exhibit symptoms commonly associated with disorders or diseases related to iron deficiency, iron uptake, and/or iron metabolism based on responses to a health-related quality of life questionnaire. Examples of iron deficiency disorders include iron deficiencies caused by insufficient dietary intake or absorption of iron. Iron deficiency disorders may be related to, for example, malnutrition, pregnancy (including the postpartum period), heavy uterine bleeding, chronic disease (including chronic kidney disease), cancer, renal dialysis, gastric by-pass, multiple sclerosis, restless leg syndrome, diabetes (e.g., Type I or Type II diabetes), insulin resistance, and attention deficit disorders.

As used herein, “transferrin saturation (TSAT)” means the ratio of serum iron to total iron-binding capacity (TIBC).

As used herein “total iron-binding capacity (TIBC)” means the total amount of iron that can be bound with serum proteins.

As used herein, “functional selectable marker” or “functional selectable marker gene” means a genetic element that provides for a growth advantage under certain conditions for progeneration of a recombinant host.

As used herein, “growth advantage” means that 90% or more of the progeny of the recombinant host comprise the genetic cassette.

As used herein, “genetic cassette” comprises a functional selectable marker gene and a gene encoding ferritin.

As used herein, “mutant” means a non-functional gene. As used herein, “non-mutant” means a functional gene.

Disclosed herein is a recombinant host comprising, or consisting essentially of, or consisting of, a recombinant gene encoding ferritin or a homologue thereof.

Also disclosed herein is a recombinant host comprising, or consisting essentially of, or consisting of, at least a first set of chromosomes and a second set of chromosomes, wherein at least one of the first set of chromosomes and the second set of chromosomes comprises a recombinant gene encoding ferritin or a homologue thereof.

Also disclosed herein is a recombinant host comprising a prototroph comprising, or consisting essentially of, or consisting of, a recombinant gene encoding ferritin or a homologue thereof.

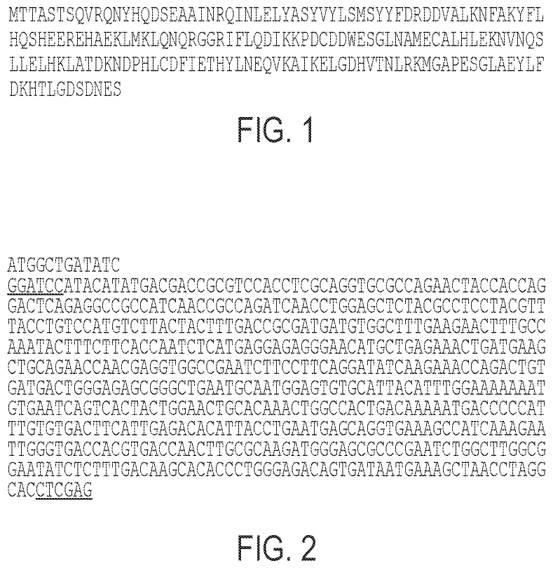

As mentioned above, the recombinant host may express ferritin (e.g., the recombinant host may comprise a gene that encodes ferritin or a homologue thereof). The gene that encodes ferritin or a homologue thereof allows the recombinant host to produce the ferritin protein through expression of the ferritin gene. Suitable ferritin may comprise mammalian or plant H-ferritin subunits and/or L-ferritin subunits. Any H-ferritin subunit from any species whatever may be used, as long as it encodes H-ferritin. The H-ferritin subunits may be mammalian H-ferritin subunits, such as, for example, human, canine, feline, bovine, equine, porcine, primate, and/or rodent. The H-ferritin subunits may be human H-ferritin (FTH1) (SEQ. ID. NO. 1; see FIG. 1; see also FIG. 2). The H-ferritin can also be a naturally occurring or synthetic homologue or variant of human H-ferritin. The H-ferritin homologue may have 80% to 100% sequence identity to human H-ferritin, such as at least 80%, 81%, 82%, 83%, 84%, 85%, 86%, 87%, 88%, 89%, 90%, 91%, 92%, 93%, 94%, 95%, 96%, 97%, 98%, or 99% sequence identity with human H-ferritin and retains the ability to bind iron and form a multi-subunit ferritin-iron complex (described below), but can be mutated to provide varying binding and disassociation strengths between the iron and the ferritin. Optionally, the ferritin may be or include L-ferritin. For example, the ferritin subunit may comprise at least 20% H-ferritin as compared to L-ferritin, such as at least 20%, 30%, 40%, 50%, 60%, 70%, 80%, 90%, or 100% H-ferritin as compared to L-ferritin. Optionally, all of the ferritin subunits (i.e., 100% of the ferritin subunits) may be H-ferritin or all of the ferritin subunits (i.e., 100% of the ferritin subunits) may be L-ferritin.

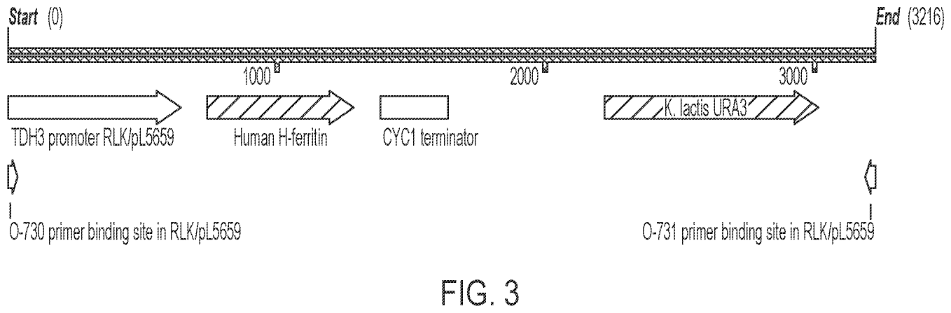

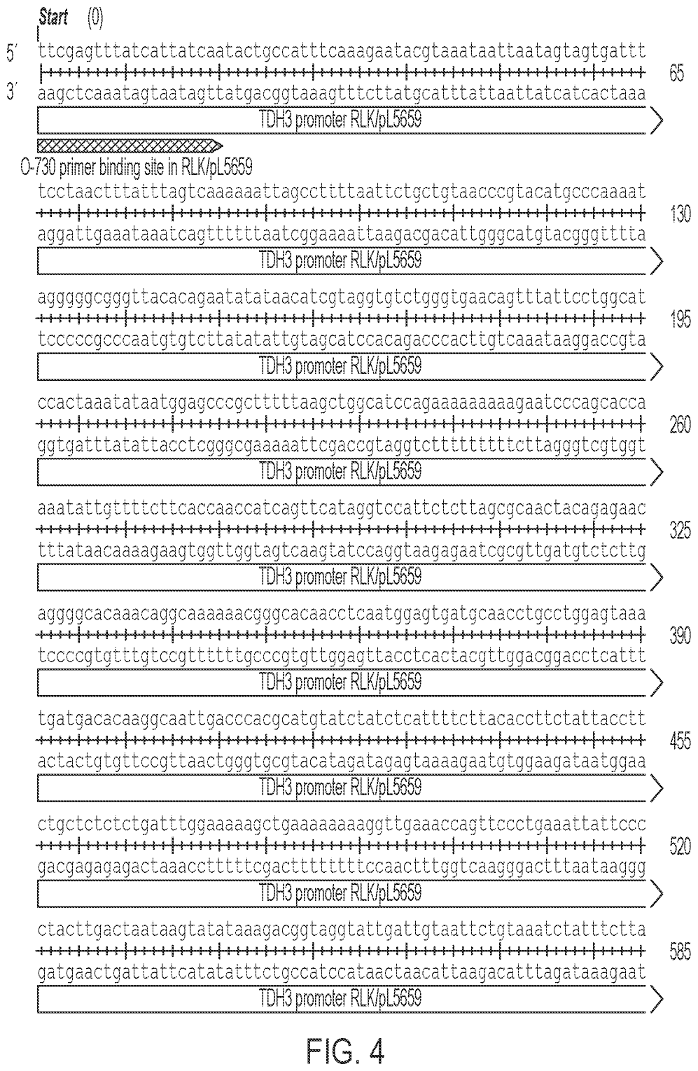

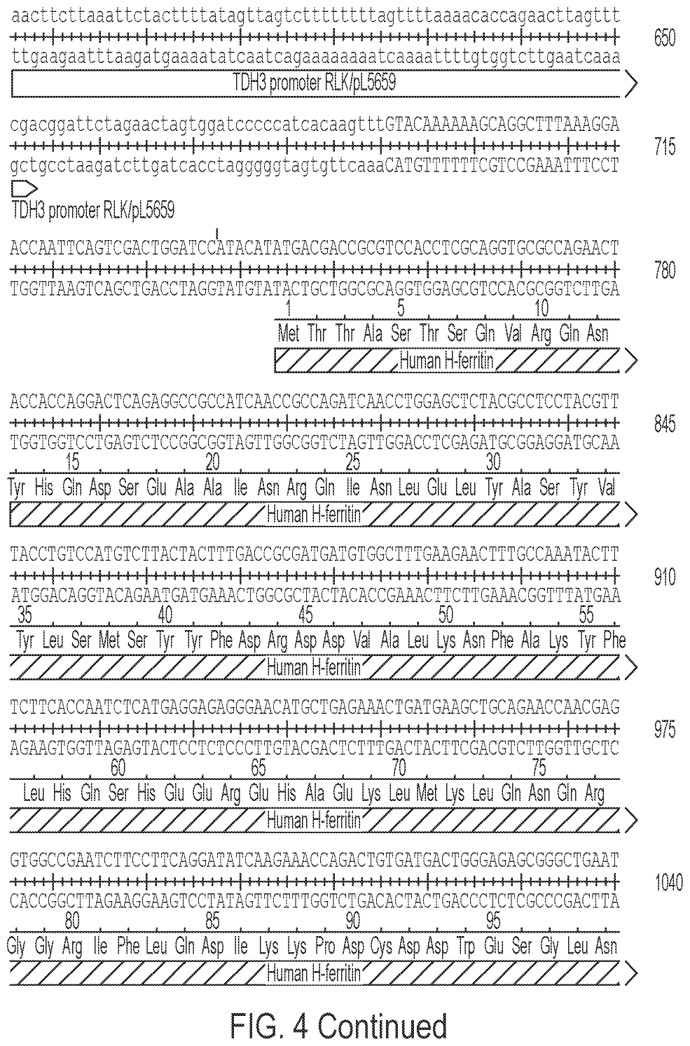

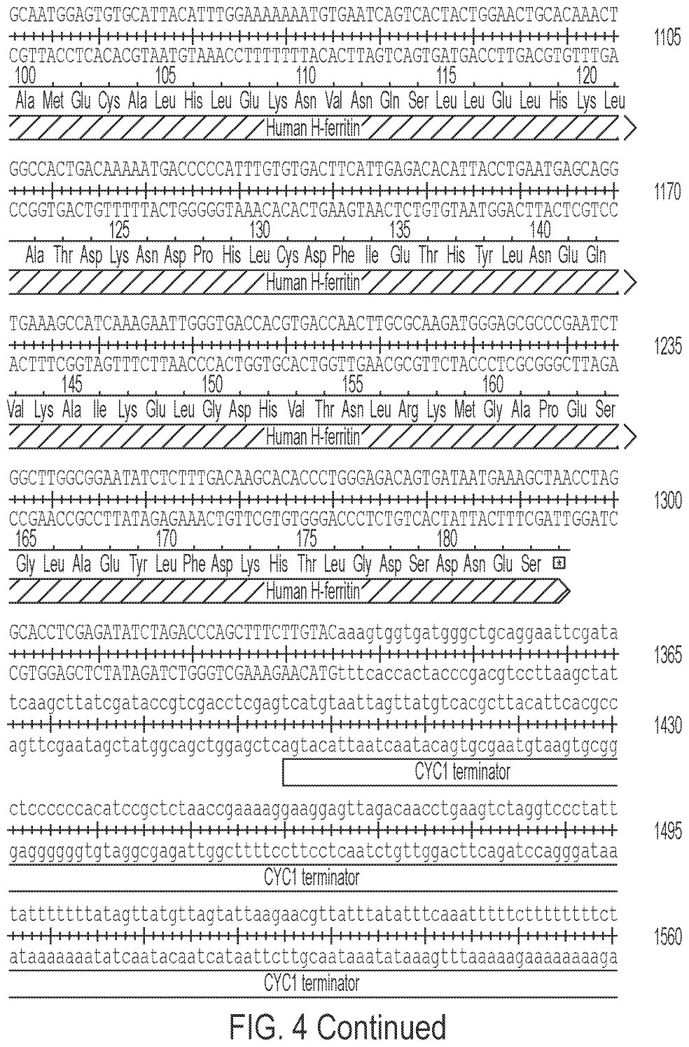

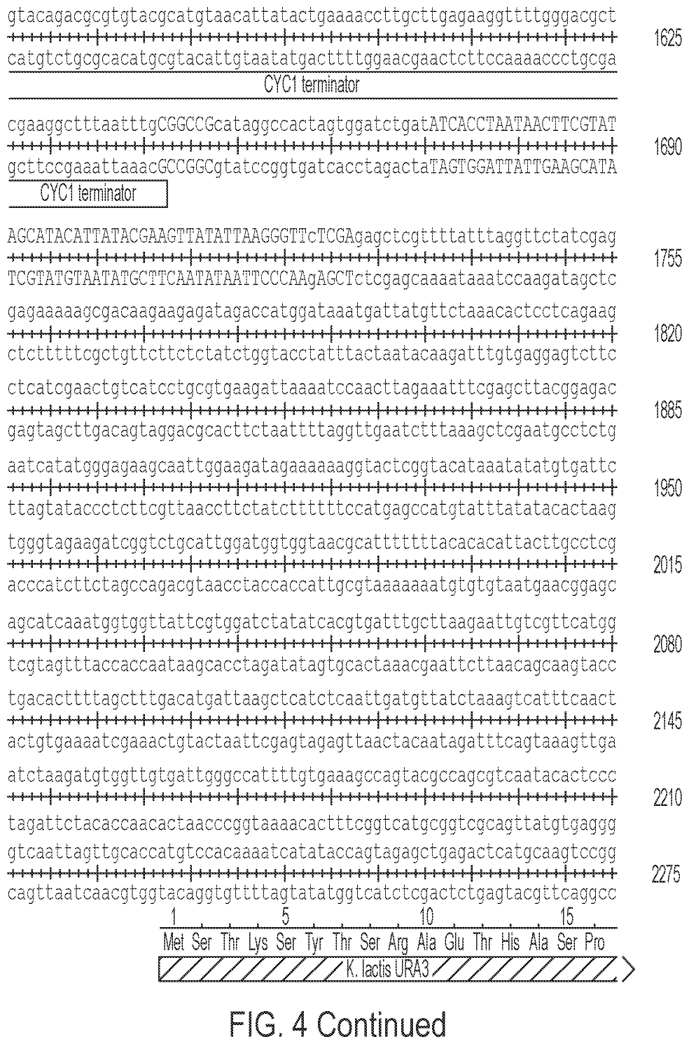

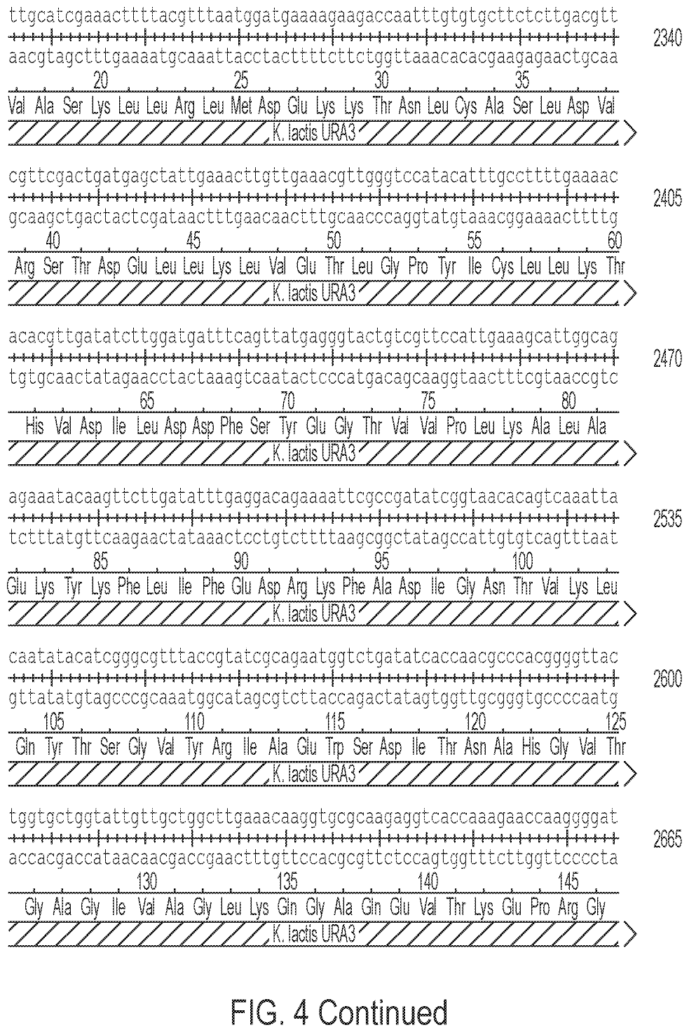

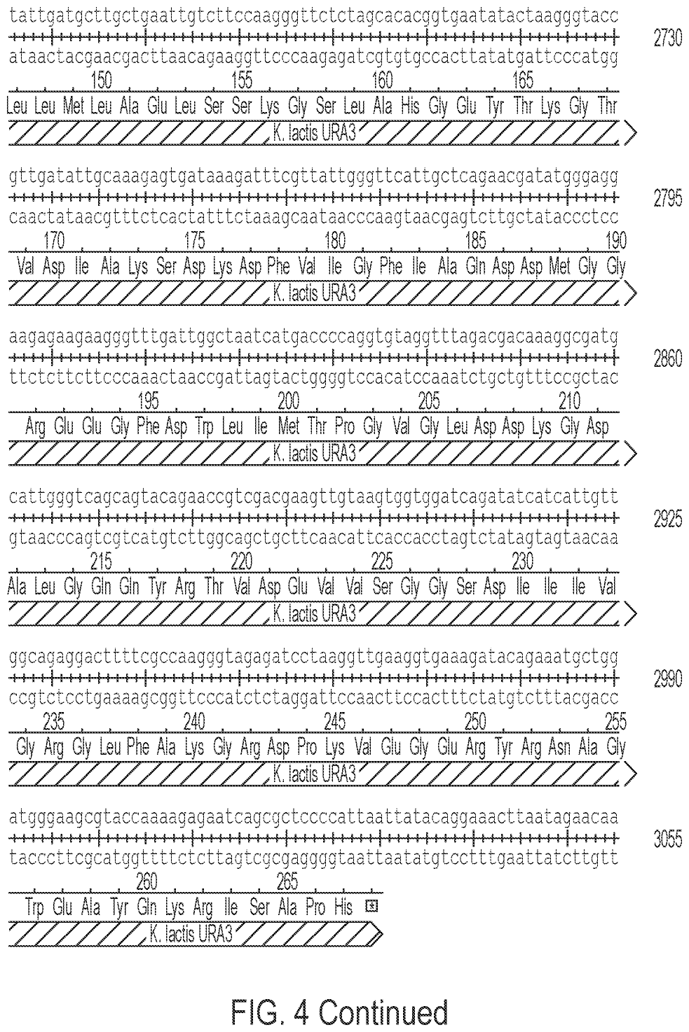

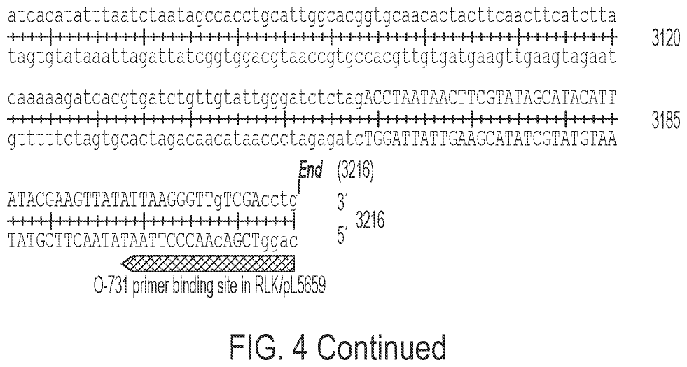

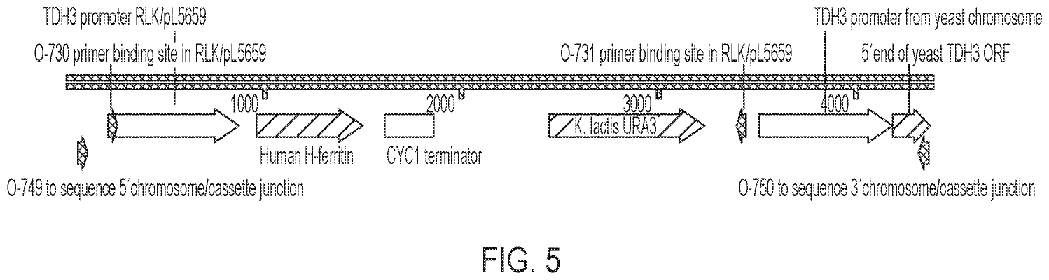

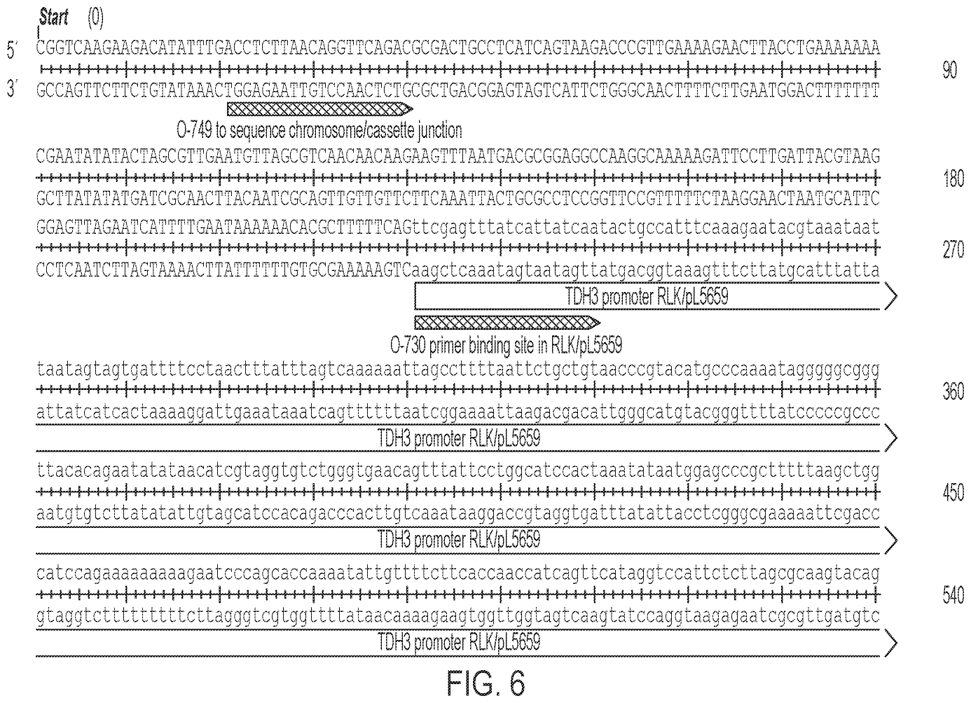

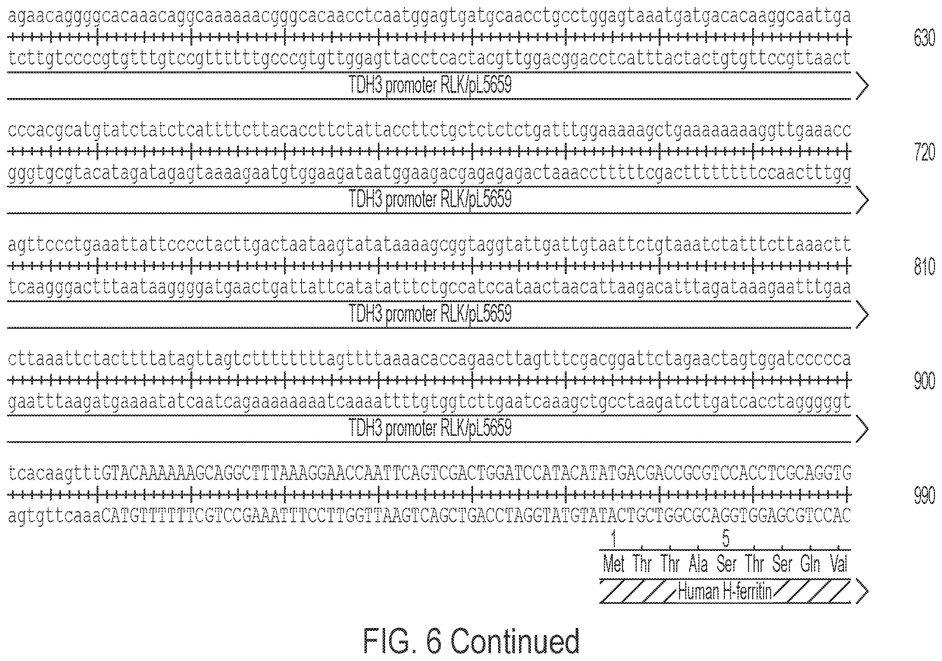

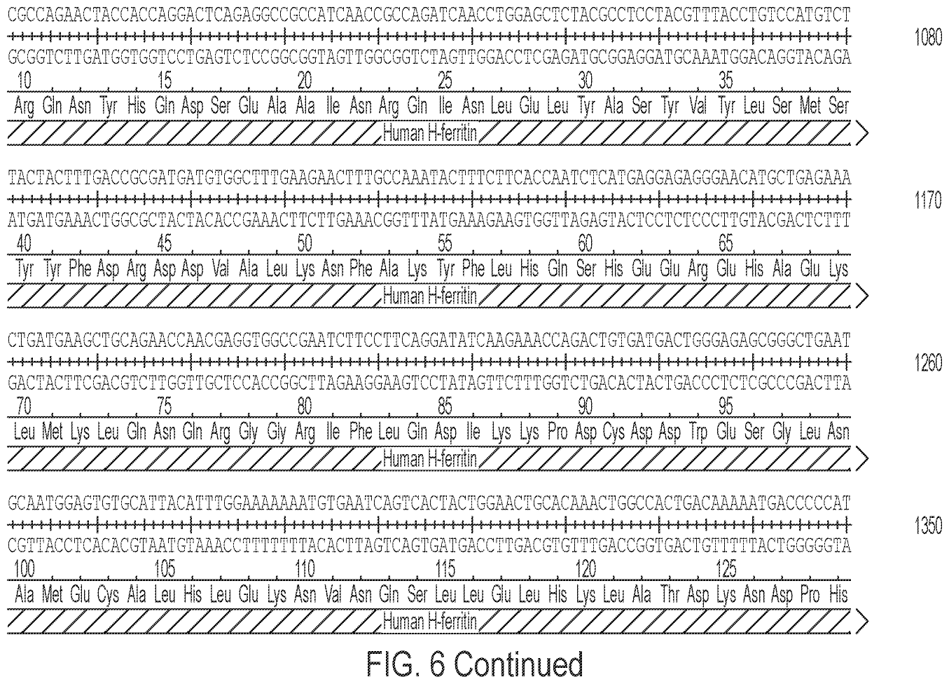

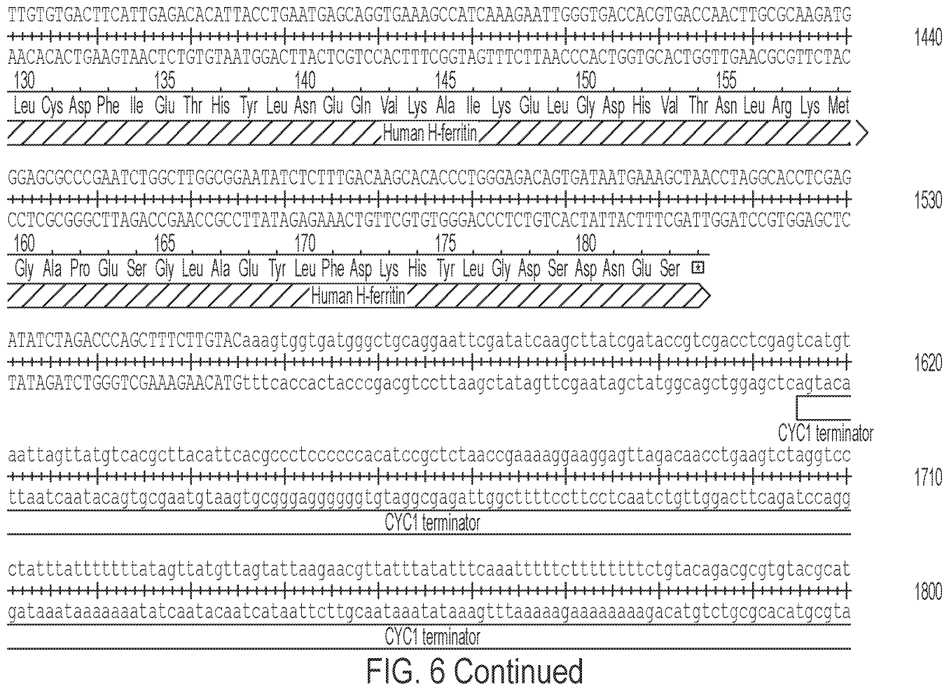

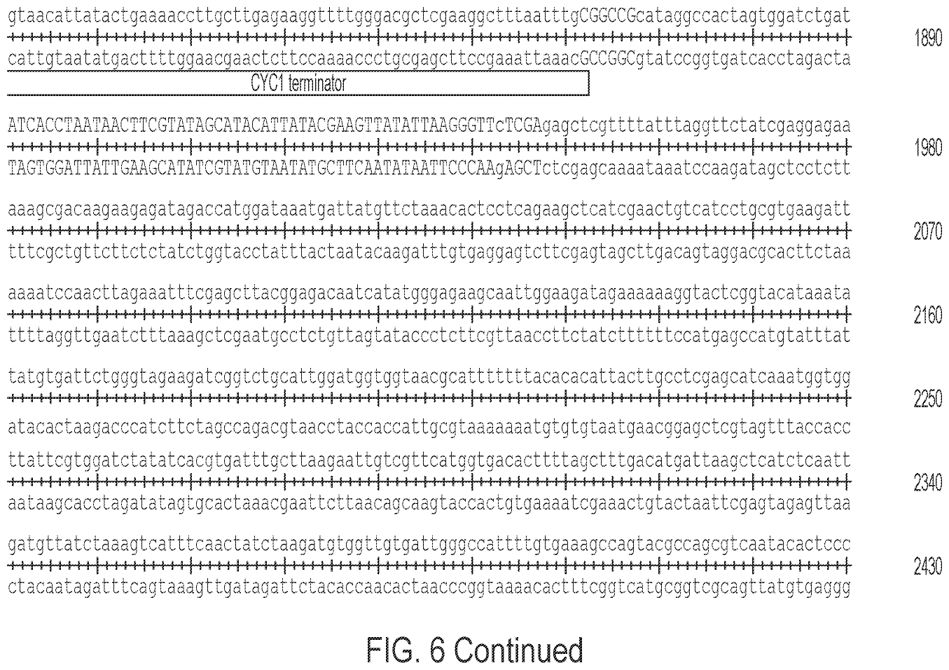

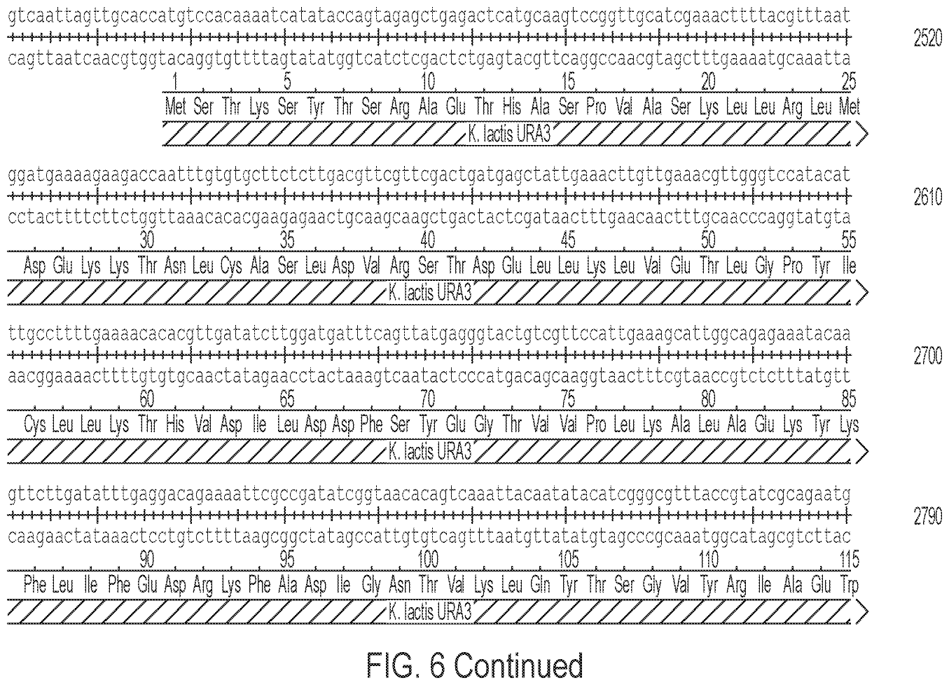

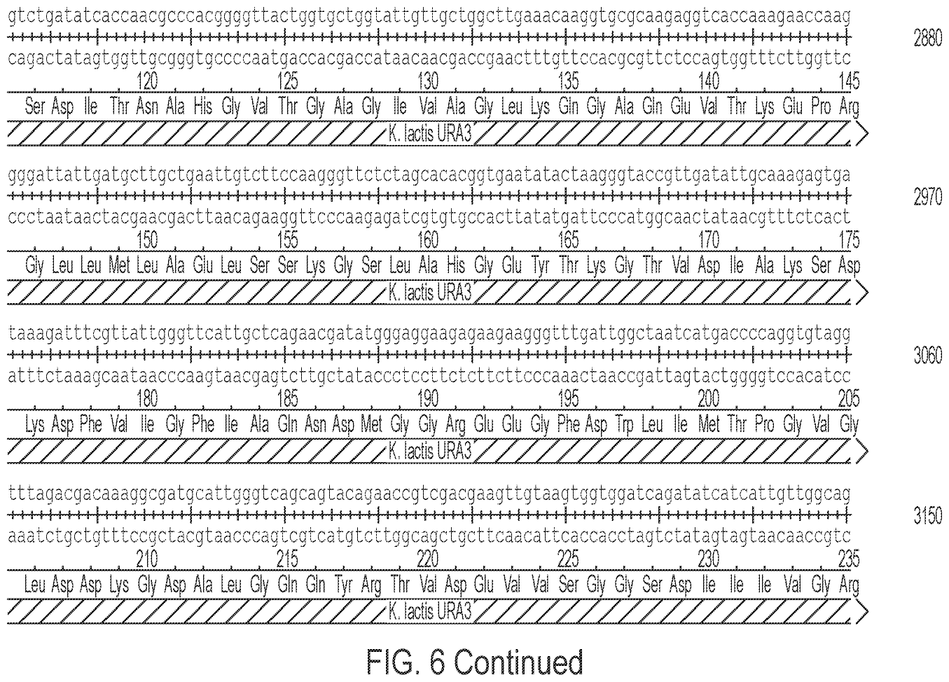

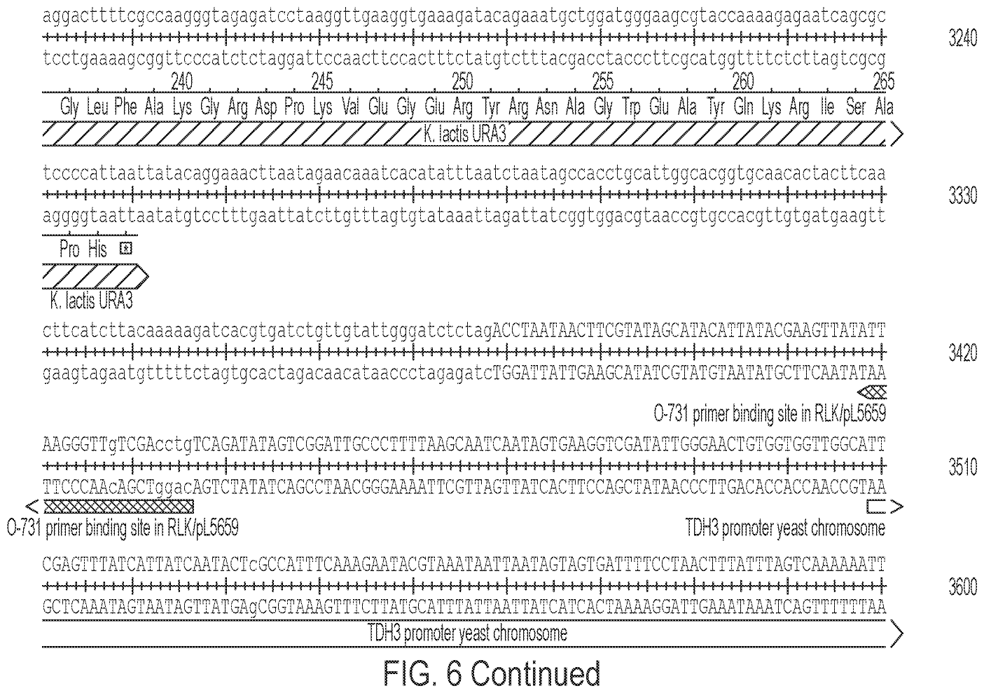

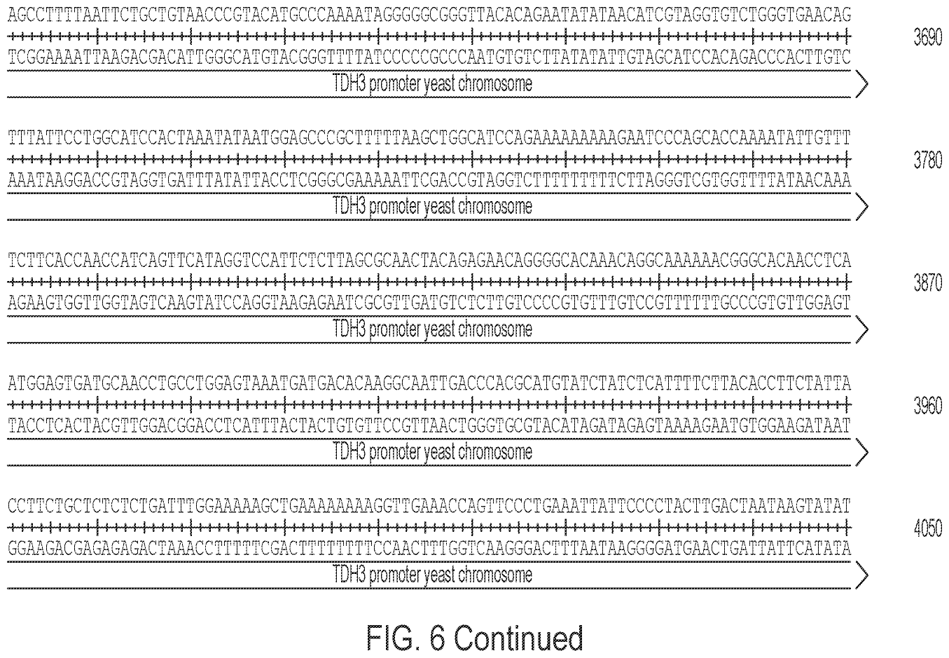

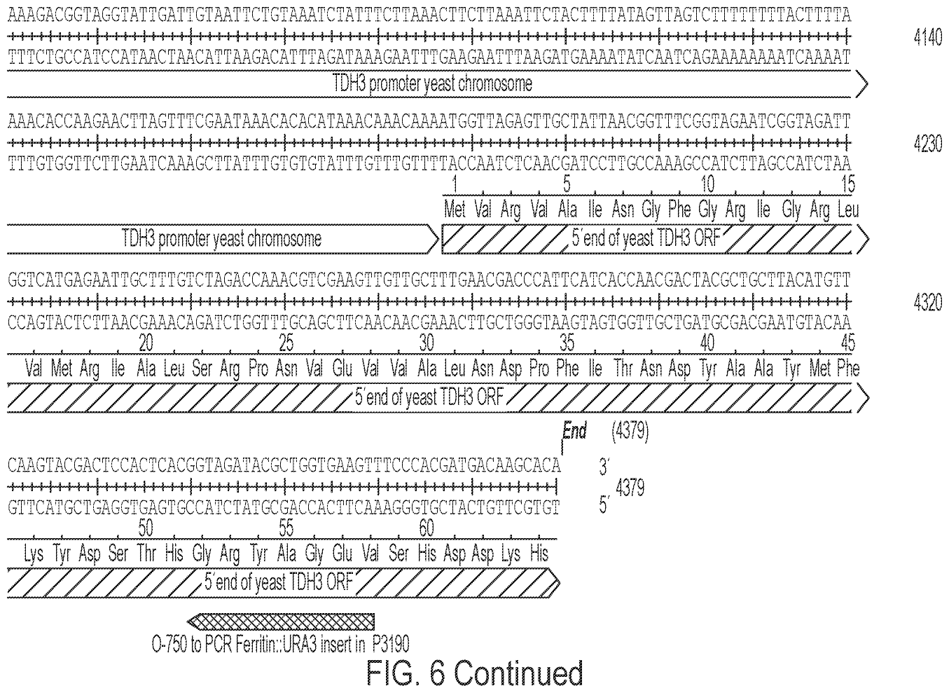

According to the present invention, the H-ferritin can be recombinant H-ferritin. For example, the H-ferritin can be human H-ferritin, or a homologue thereof, produced in a microbial strain comprising a polynucleotide sequence encoding the H-ferritin under the control of an appropriate promoter. (See SEQ ID NO:3; see also FIGS. 3 and 4). The recombinant gene encoding ferritin may be extra-chromosomal (episomal) or may be chromosomally integrated. (See SEQ ID NO:4; see also FIGS. 5 and 6).

The recombinant host can be prototrophic. The prototrophic recombinant host can be either diploid, polyploid, or aneuploid. Examples of suitable diploid recombinant hosts useful in the present invention include, but are not limited to, a fungus, an alga, a protozoan, a microscopic helminth, a microorganism, or combinations thereof. Examples of suitable polyploid or aneuploid recombinant hosts useful in the present invention include, but are not limited to, a fungus a plant, or combinations thereof. For example, recombinant microbe strains suitable for nutritional supplementation of iron can store iron in a form having high bioavailability for mammals, including humans, such as those that meet the Generally Regarded As Safe (GRAS) requirements for human consumption. Other microbes that can be used in processes to produce therapeutic compounds also may be used in the composition of the present invention. The fungus may be, for example, a yeast. Non-limiting examples of yeast include various species of the genus Saccharomyces, Schizosaccharomyces, Kluyveromyces, or Pichia. Non-limiting examples of algae include various species of the genus Chlamydomonas. The recombinant host may contain impurities that may contribute to the weight of a composition in the present invention, but these weights are excluded from the total dry matter weight of the composition.

As noted above, the recombinant host may be diploid and prototrophic. When the recombinant host is diploid and prototrophic, a non-mutant copy of each gene is present on at least one of the two sets of chromosomes. This allows a diploid recombinant host to produce all of the nutrients needed to be self-sustaining from inorganic materials, thus making it prototrophic.

As shown in FIGS. 3 and 5, in examples, the H-ferritin gene may be stably integrated into the set of chromosomes of a haploid recombinant host. In other examples, a first set of chromosomes of a diploid, polyploid, or aneuploid recombinant host may comprise the H-ferritin gene stably integrated. For purposes of this application, the terms “a first set of chromosomes” and “a second set of chromosomes” are used as a matter of convenience and do not indicate a particular order or limitation on the number of chromosomes (i.e., there may be two, or three, or more complete or partial sets of chromosomes which are referred to herein for convenience as “the second set of chromosomes”). In still other examples, both sets of chromosomes of a diploid recombinant host or at least two chromosomes of a polyploid or aneuploid may comprise the H-ferritin gene stably integrated. The H-ferritin gene can be closely linked to any functional selectable marker gene, making up the genetic cassette. Suitable functional selectable marker genes will be known by those skilled in the art, for example, URA3, LEU2, TRP1, HIS3, or MET15. For example, the H-ferritin gene can be closely linked to a URA3 gene. The copy of the functional selectable marker gene that exists in the chromosome of the recombinant host can be a mutant of the functional selectable marker gene, making it non-functional. As will be understood by those of skill in the art, this promotes selection for the haploid comprising the genetic cassette of the first haploid and the second haploid can be a mutant URA3 gene. For clarity, in addition to the H-ferritin gene and the functional selectable marker gene, the genetic cassette may further comprise other components known to those skilled in the art, including, for example, promoters and terminators.

The recombinant host comprising the H-ferritin gene can comprise at least one diploid cell, polyploid cell, or aneuploid cell. A haploid comprises a single full set of chromosomes. When the recombinant host is a diploid, two haploids of opposite mating types mate to form the diploid recombinant host. One of the chromosomes in the first set of chromosomes of the recombinant diploid host can comprise the recombinant H-ferritin gene. The recombinant H-ferritin gene may be part of a chromosomally integrated genetic cassette. The recombinant H-ferritin gene encodes an amino acid sequence for the production of H-ferritin. The H-ferritin coding sequence may be placed under the control of an appropriate promoter in a genetic cassette to produce high enough levels of the iron-storage protein for the recombinant host to serve as a suitable vehicle for iron supplementation. Suitable promoters are known in the art and include promoters that induce a high level of constitutive expression and promoters whose expression can be regulated by environmental conditions. For example, in a yeast recombinant host an appropriate constitutive promoter may be the yeast TDH3 transcription promoter. For example, in a yeast recombinant host an appropriate regulatable promoter may be the yeast GAL1 promoter. In addition, the genetic constitution of the recombinant host can be further manipulated to achieve a variety of potentially advantageous outcomes. For example, proteolysis may be manipulated to enhance the stability of the iron-storage protein. Iron transport mechanisms can be manipulated to achieve desirable outcomes, such as altering the iron concentration in specific cellular compartments, including but not limited to those of the cell surface, the vacuole, or the mitochondria. The iron content of the recombinant host may be regulated by adding known amounts of an iron compound to the medium in which the recombinant host is grown. Using the recombinant host, iron supplementation for humans and other animals can be accomplished by any of a number of means including, but not limited to, consumption or ingestion of the recombinant host. The recombinant host may be grown specifically for the purpose of iron supplementation or may be the by-product of another process (e.g., fermentation). In examples, a diploid, polyploid, or aneuploid recombinant host may have only the typical nutritional requirements of prototrophic yeast. That is, auxotrophic requirements of a set of chromosomes encoding ferritin due to genetic mutations are complemented by the presence of a non-mutant copy of the gene in a second set of chromosomes. Similarly, auxotrophic requirements of the second set of chromosomes due to genetic mutations are complemented by the presence of a non-mutant copy of the gene in the first set of chromosomes.

In examples, the second set of chromosomes may not express ferritin. The second set of chromosomes may additionally not express a functional selectable marker. That is, the second set of chromosomes may not comprise the genetic cassette. For example, the first set of chromosomes may comprise a mutant LEU2 gene, which is involved in synthesis of leucine, an amino acid essential for the production of protein. The second set of chromosomes may comprise a non-mutant LEU2 gene, allowing the diploid, polyploid, or aneuploid comprising at least the first set of chromosomes and the second set of chromosomes to synthesize leucine. The ability of the second set of chromosomes to synthesize leucine compensates for the inability of the first set of chromosomes to synthesize the amino acid. As a result, the diploid, polyploid, or aneuploid recombinant host is able to produce all of the normal nutritional materials prototrophic yeast synthesize (i.e., the recombinant host is a prototroph). That is, a non-mutant copy of each gene is present in at least one of the two homologous chromosomes in a diploid recombinant host, at least one of three or more homologous chromosomes in a polyploid recombinant host, or at least one of the at least two sets of chromosomes in an aneuploid or aneuploid recombinant host.

For example, the first set of chromosomes may comprise a mutant URA3 gene, which is involved in synthesis of uracil, a nucleobase essential for the synthesis of ribonucleic acid (RNA). The second set of chromosomes may comprise a non-mutant URA3 gene, allowing the second set of chromosomes to synthesize uracil. The ability of the second set of chromosomes to synthesize uracil compensates for the inability of the first set of chromosomes to synthesize the nucleobase. As a result, the diploid or polyploid recombinant host can produce all of the normal materials prototrophic yeast synthesize (i.e., the recombinant host is a prototroph).

In other examples, the first set of chromosomes and the second set of chromosomes may encode ferritin. Both sets of chromosomes may additionally encode a functional selectable marker gene. That is, both sets of chromosomes comprise the genetic cassette. When both sets of chromosomes express the genetic cassette, the genetic cassette can be at different loci, so that each set of chromosomes has different mutations. For example, the first set of chromosomes may comprise a mutant LEU2 gene and a non-mutant URA3 gene, while the second set of chromosomes comprises a non-mutant LEU2 gene and a mutant URA3 gene.

The present invention also discloses a composition comprising one of the recombinant hosts disclosed herein. In an example, the composition may comprise, or consist essentially of, or consist of, a recombinant host comprising at least a first set of chromosomes and a second set of chromosomes, wherein at least one of the first set of chromosomes and the second set of chromosomes comprises a recombinant gene encoding ferritin or a homologue thereof. In another example, the composition may comprise, or consist essentially of, or consist of, a recombinant host comprising a prototroph comprising a recombinant gene encoding ferritin or a homologue thereof. The composition may further comprise iron. The ferritin expressed by the recombinant host and the iron may form a ferritin-iron complex. A source of the iron may be an iron salt, an organic iron complex, an elemental iron nanoparticle, or combinations thereof. An example of a suitable iron salt includes, but is not limited to, iron sulfate. Examples of suitable iron complexes include, but are not limited to, iron polymer complexes, iron carbohydrate complexes, and iron aminoglycosan complexes. These organic-iron complexes may be commercially available and/or can be synthesized by methods known in the art. Suitable non-limiting examples of iron carbohydrate complexes include iron saccharide complexes, iron oligosaccharide complexes, and iron polysaccharide complexes, such as iron carboxymaltose, iron sucrose, iron polyisomaltose (iron dextran), iron polymaltose (iron dextrin), iron gluconate, iron sorbital, and iron hydrogenated dextran, which may be further complexed with other compounds, such as sorbitol, citric acid, and gluconic acid (for example, iron dextrin-sorbitol-citric acid complex and iron sucrose-gluconic acid complex), and mixtures thereof. Suitable non-limiting examples of iron aminoglycosan complexes include chondroitin sulfate, iron dermatin sulfate, and/or iron keratan sulfate, each of which may be further complexed with other compounds, and mixtures thereof. Examples of iron aminoglycosan complexes include but are not limited to iron hyaluronic acid, iron protein complexes, and mixtures thereof.

The iron may be present in the composition in an amount of at least 1% by weight on a dry matter basis of the recombinant host expressing ferritin and the iron, such as at least 3% by weight, such as at least 5% by weight, such as at least 5.5% by weight. The iron may be present in an amount of no more than 15% by weight on a dry matter basis of the recombinant host expressing ferritin and the iron, such as no more than 10% by weight, such as no more than 9.5% by weight, such as no more than 8% by weight. The iron may be present in the composition in an amount of 1% by weight to 15% by weight on a dry matter basis of the recombinant host expressing ferritin and the iron, such as 3% to 12%, such as 3% to 8%, such as 5% to 10%, such as 5.5% to 9.5% by weight.

The composition may comprise intracellular and/or extracellular iron that is not complexed with the ferritin or homologue thereof.

According to the present invention, in some instances, at least 5% of the iron may be complexed with the ferritin, such as at least 10% of the iron may be complexed with the ferritin, such as at least 15%, such as at least 20%, such as at least 30%, such as at least 40%, such as at least 50%, such as at least 60%, such as at least 70%, such as at least 80%, such as at least 90%, such as 100%. According to the present invention, in some instances, no more than 95% of the iron may be complexed with the ferritin, such as no more than 90%. According to the present invention, 5% to 100% of the iron may be complexed with the ferritin, such as 10% to 100%, such as 30% to 95%, such as 50% to 90%, such as 60% to 90% such as 75% to 90%.

The composition of the invention may optionally further comprise a second recombinant host. As used herein, the term “second” with respect to a recombinant host refers to a separate and distinct recombinant host and does not necessarily mean that only two recombinant hosts are present. The second recombinant host may comprise any of the recombinant hosts discussed above. The second recombinant host may comprise a recombinant host that expresses ferritin, a recombinant host that does not express ferritin, or a combination thereof. The second recombinant host may additionally or alternatively comprise a probiotic, a prebiotic, or combinations thereof.

Any of the compositions described herein may be included in an ingestible item. In examples, the recombinant host expressing ferritin may be included in the ingestible item. For example, the ingestible item may be a medical food, a food, a food ingredient, a dietary supplement, a pharmaceutical composition, or combinations thereof. In other examples, any of the compositions described herein may be in the form of a suppository. In other examples, any of the compositions described herein may be a dietary or nutritional supplement. In examples, the recombinant host expressing ferritin may be included in the dietary supplement or nutritional supplement.

In other examples, any of the compositions described herein may be included in a pharmaceutical composition. The pharmaceutical composition may be administered for the treatment of, for example, an iron deficiency disorder, such as anemia. The pharmaceutical composition may be administered whenever a high dosage of iron would be beneficial to the treatment of iron deficiency.

The compositions described herein may be in the form of a dry powder, a dispersion of the dry powder in a liquid, a suspension of the dry powder in a liquid, suppository, foam enema, liquid enema, or the like and may be formulated in such a manner as to be administered orally or rectally. The compositions of the present invention may include a pharmaceutically acceptable carrier or diluent (described herein) to form a solution, dispersion, emulsion, microemulsion, suspension, syrup, elixir or the like such that the materials may be swallowed. pH adjusters (i.e., acids or bases) may be included to adjust pH to the appropriate level, and/or bacterial and antifungal agents may be included to prevent the action of microorganisms. Pharmaceutical compositions also may include formulations that control or slow the release of the agent in the body. In some instances, the pharmaceutical composition may be included in a dispenser, such as a syringe, dosing vial, and the like.

Examples of ingestible diluents or carriers are sugars such as monosaccharides, disaccharides, and the like; excipients such as cocoa butter and waxes; oils such as peanut oil, cottonseed oil, safflower oil, sesame oil, olive oil, corn oil, and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol; phosphate buffer solutions; other non-toxic compatible lubricants such as sodium lauryl sulfate and magnesium stearate; coloring agents; releasing agents; coating agents; preservatives and antioxidants according to the judgment of the formulator.

The compositions described herein may be expelled from a pressurized container, or may be in the form of powders, granules, or lozenges. The composition may further comprise at least one binder and/or at least one filler. Examples of suitable binders and fillers include, but are not limited to, magnesium stearate, microcrystalline cellulose, cellulose gel, cellulose gum, carboxymethyl cellulose, wood pulp, soy lecithin, glycine, monosodium glutamate, vegetable protein, seaweed or extract, carrageenan, or combinations thereof.

As used herein, the term “pharmaceutically acceptable” means acceptable for use in the pharmaceutical and veterinary arts, compatible with other ingredients of the formulation, and not toxic or otherwise unacceptable and commensurate with a reasonable benefit/risk ratio. As used herein, “pharmaceutically acceptable carrier” includes any and all solvents, diluents, or other liquid vehicle, dispersion or suspension aids, surface active agents, isotonic agents, thickening and emulsifying agents, stabilizers, preservatives, solid binders, lubricants, and the like, as suited to the particular dosage form desired. Remington's Pharmaceutical Sciences 19th Ed. by Gennaro, Mack Publishing, Easton, P A 1995 provides various carriers used in formulating pharmaceutical compositions and known techniques for the preparation thereof.

The skilled artisan understands that various factors influence the dosage required to treat a patient effectively, and that accordingly the dosage and administration may be chosen by the attending physician in view of the patient to be treated and may be adjusted for sufficient levels of the active agent(s) or to maintain the desired effect. Additional factors that may be taken into account include the severity of the disease state, e.g., intermediate or advanced stage of disease; age, weight, gender and overall health of the patient; diet, time and frequency of administration; form of iron deficiency; route of administration; drug combinations; reaction sensitivities; prior treatments; and tolerance/response to therapy. Pharmaceutical compositions may be administered, for example, every 30 minutes, hourly or daily; multiple times per day; weekly, multiple times per week; bi-weekly; monthly; and the like.

The active agents of the invention may be used to treat any of the diseases, disorders, or the like, disclosed herein, and may be administered as an effective dose appropriate for the patient or subject to be treated. As described above, the effective dose of the compositions of the present invention may be decided by the attending physician within the scope of sound medical judgment and experience. For the active agent, the effective dose may be estimated initially in cell culture assays or in animal models such as mice, rats, rabbits, dogs, or pigs. Animal cell models may be used to achieve or determine a desirable concentration and total dosing range and route of administration, which may be used to determine a useful range of dosage and routes for administration in humans. Further, clinical studies and individual patient response may determine the recommended effective dose.

An effective dose of one of the compositions described herein may be administered at a dosage level of at least 13 mg of iron per dose, such as at least 50 mg per dose, such as at least 75 mg per dose, such as at least 100 mg per dose or more. An effective dose of one of the compositions described herein may be administered at a dosage level of no more than 1000 mg per dose, such as no more than 700 mg per dose, such as no more than 300 mg per dose. An effective dose of one of the compositions described herein may be administered at a dosage level of 13 mg to 1000 mg of iron per dose, such as 50 mg to 700 mg per dose, such as 75 mg to 300 mg per dose, such as 100 mg to 300 mg per dose. As described herein, the dosage level may be administered as a single dose administered to the subject or patient, or through multiple administrations that achieve the dosage level over the course of a day. The dosage level may also be a total amount of iron administered for multiple times per week, weekly, bi-weekly, or monthly administration divided by the number of days between administration, wherein the dose administers iron in a dosage level described above on a per day average.

As described above, the methods described herein generally include the administration of any of the compositions described herein to a subject. The method may comprise, or consist essentially of, or consist of, administering a composition comprising, or consisting essentially of, or consisting of, (a) a recombinant host expressing ferritin and (b) iron in an amount of at least 1% by weight on a dry matter basis of the recombinant host expressing ferritin and the iron; wherein at least 60% of the iron is complexed with the ferritin. For example, disclosed is a method for treating a subject comprising administering to the subject any of the compositions described herein. The administering may comprise administering to the subject an effective amount of at least one of the compositions described herein. As used herein, the term “effective amount” or “effective dose” is an amount of the composition indicated for treatment (i.e., modulating, ameliorating, or preventing symptoms or conditions of iron deficiency) while not exceeding any amount which may cause adverse effects. An effective dose may increase or decrease over the course of treatment. Methods for evaluating the effectiveness or toxicity of effective treatments are known to those of skill in the art, e.g., ED50 (the dose is effective in 50% of the population) and LD50 (the dose is lethal to 50% of the population). The dose ratio of toxic to effective effects is the therapeutic index, and it is expressed as the ratio, LD50/ED50. Notably. ED50 and LD50 may vary with age or condition of the subject.

The composition may be administered as a single dose or as multiple doses (i.e., first, second, third, etc. doses) administered contemporaneously or sequentially, such that administration of a first dose of the composition is followed by administration of a second dose of the composition, or vice versa. When the first and second doses are administered sequentially, the method may comprise waiting a period of time between the administration of the doses. First, second, third, etc. doses may comprise the same or different amounts of iron. As used herein, the term “sequentially” refers to a treatment protocol in which administration of a first dose of a composition of the present invention follows administration of a second dose of a composition of the present invention. As used herein, the term “contemporaneously” refers to administration of a first dose of a composition of the present invention and administration of a second dose of a composition of the present invention, wherein the first and second doses are separate and are administered at substantially the same time.

According to the present invention, the iron-deficiency of a subject may be treated by administering to the subject any of the compositions described herein, such as administering an effective amount of any of the compositions described herein. For example, a method of treating a subject may comprise, or consist essentially of, or consist of, administering to the subject a composition comprising (a) a recombinant host expressing ferritin and (b) iron in an amount of at least 1% by weight based on dry weight of the recombinant host expressing ferritin and the iron. The administering may comprise oral administration or rectal administration. The subject may be determined to have at least one of the following prior to the administering: functional iron deficiency; iron deficiency; anemia; iron deficiency anemia; or a gut microbiome disorder. The subject may not have iron deficiency. A suitable subject would be any subject that would benefit from taking the composition. The treating may comprise dietary management.

Whereas specific aspects of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of the invention which is to be given the full breadth of the claims and aspects appended and any and all equivalents thereof.

It will be appreciated by skilled artisans that numerous modifications and variations are possible in light of the above disclosure without departing from the broad inventive concepts described and exemplified herein. Accordingly, it is therefore to be understood that the foregoing disclosure is merely illustrative of various exemplary aspects of this application and that numerous modifications and variations can be readily made by skilled artisans which are within the spirit and scope of this application and the accompanying claims.

### EXAMPLES

### Example 1

Twenty-day old male Sprague Dawley rats (Envigo) were housed 1 per cage in hanging wire cages in a temperature (23±2° C.) and humidity (40%) controlled room maintained on a 12:12 hr light/dark cycle (lights on 0600 to 1800). Rats were fed an iron deficient diet (“ID diet”) of 2 ppm iron or supplement diets ad libitum as indicated. The ID diet was prepared following the recipe of the American Institute of Nutrition (AIN)-93G diet with cornstarch as the sole source of carbohydrate and without the addition of iron. Iron levels for all diets were verified using atomic absorption spectrometry after wet digestion with nitric acid (Perkin Elmer). All experimental protocols were in accordance with The National Institutes of Health Animal Care guidelines and were approved by The Pennsylvania State University Institutional Animal Care and Use Committee.

Rats were fed an ID diet for 26 days (P21-P47), which produced anemia (mean hematocrit and hemoglobin levels were 5.3±0.2 and 16.2±0.1%, respectively). At P47, rats were then divided into 2 dietary groups (n=3-5/group) balanced by body weight. Dietary group 1 was fed a diet comprising a haploid yeast-ferritin complex (53 μg iron/g diet; n=4). The haploid yeast-ferritin complex comprises a single chromosome that comprises the chromosomally integrated ferritin expression cassette in FIG. 5. The haploid yeast-ferritin complex is auxotrophic. Dietary group 2 was fed a diet comprising a diploid yeast-ferritin complex as described in the present application (49 μg iron/g diet; n=3). The diploid yeast-ferritin complex comprises two chromosomes—a first chromosome comprises the chromosomally integrated ferritin expression cassette in FIG. 5 and a second chromosome is absent the chromosomally integrated ferritin expression cassette. The diploid yeast-ferritin complex is prototrophic. Rats were fed the assigned diet for 14 days, and food consumption was monitored throughout the study.

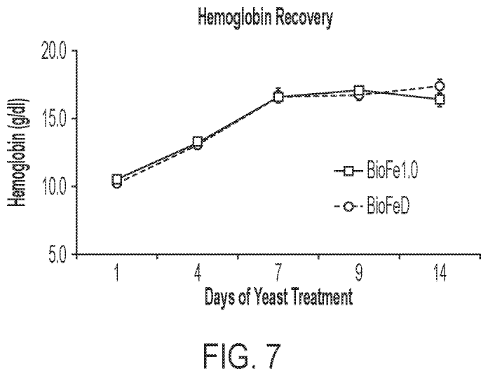

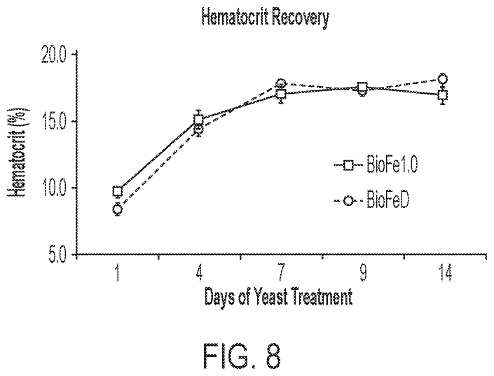

The results shown in FIG. 7 and FIG. 8 show that the diploid yeast-ferritin complex provided comparable hemoglobin recovery and hematocrit recovery, respectively, as the haploid yeast-ferritin complex.

### Example 2

Preparation of Haploid S. cerevisiae Yeast

Haploid S. cerevisiae was grown in synthetic complete (SC) medium lacking uracil with 6 mM FeSO₄ at 30° C. with shaking for two days. Cells were collected by centrifugation and washed once with sterile water. Following resuspension, cells were incubated at 65° C. for 30 min. to pasteurize. Cells were then washed two additional times with sterile water to remove unincorporated iron and lyophilized.

Preparation of Diploid S. cerevisiae Yeast

Diploid S. cerevisiae was grown in synthetic complete (SC) medium lacking uracil with 6 mM FeSO₄ at 30° C. with shaking for two days. Cells were collected by centrifugation and washed once with sterile water. Following resuspension, cells were incubated at 65° ° C. for 30 min. to pasteurize. Cells were then washed two additional times with sterile water to remove unincorporated iron and lyophilized.

### Analysis of Haploid and Diploid Yeast Samples

To analyze the haploid and diploid yeast samples for ferritin, an aliquot of the yeast preparation was viewed with electron microscopy. To further demonstrate the presence of FHT1, a western blot analysis was performed on the yeast lysate. The samples were run on a 4-20% Tri-Glycine SDS gel. Following this, it was transferred to a PVDF membrane and probed first with an antibody against H-ferritin (1:500, Cell signaling) followed by a secondary antibody (1:5000, GE healthcare). To determine if the ferritin in the yeast contained iron, a 25 μl of the yeast lysate was run on a 4-20% Tris-Glycine SDS gel (BioRad) and used to assess iron using Perls' strain. Briefly, the gel was rinsed once with de-ionized water followed by incubation with a solution consisting of two parts potassium ferricyanide (Sigma Aldrich, cat #702587) and one part 10% HCl solution for 30 min. The gel was subsequently washed with deionized water several times to remove background and observed for a blue Perls' reaction.

In order to analyze the iron content of the yeast samples, yeast was digested in nitic acid and the iron amounts were determined using atomic absorption spectrophotometry.

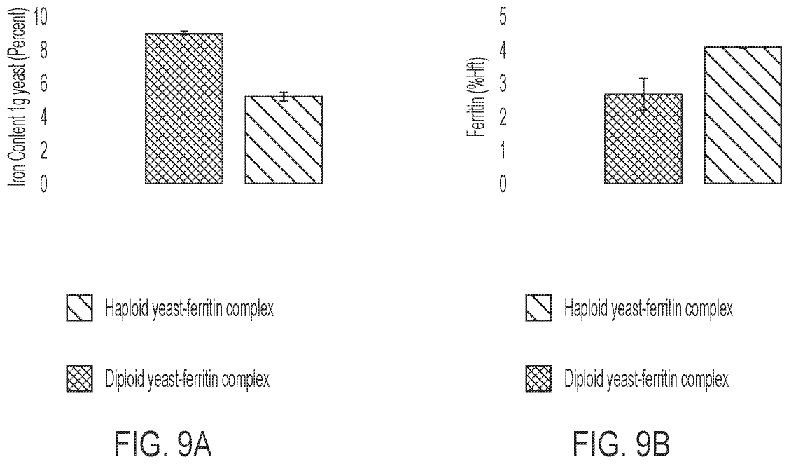

The results shown in FIG. 9A compared the iron content of the haploid yeast sample and the diploid yeast sample based on weight percent of a 1 g sample of yeast. As shown, the diploid yeast sample had higher iron levels (9%) compared to the haploid yeast sample (5%). The results shown in FIG. 9B compared the H-ferritin content of the haploid yeast sample and the diploid yeast sample. As shown, the haploid yeast sample had a greater amount of H-ferritin (4.12%) compared to the diploid yeast sample (2.76%).

Thus, despite the fact there was less H-ferritin in the diploid yeast sample, there was more iron as compared to the haploid yeast sample. Additionally, despite the differences, the haploid yeast and the diploid yeast performed equally well in in vivo studies. This suggests that the diploid yeast was more efficient at absorbing iron, while still maintaining performance.

We claim:

1. A recombinant host comprising:
at least a first set of chromosomes and a second set of chromosomes, wherein at least one of the first set of chromosomes and the second set of chromosomes comprise a recombinant gene encoding ferritin or a homologue thereof.

2. The recombinant host of claim 1, wherein the first set of chromosomes and the second set of chromosomes comprise a diploid, a polyploid and/or an aneuploid.

3-4. (canceled)

5. The recombinant host of claim 1, wherein the recombinant host is prototrophic.

6. The recombinant host of claim 1, wherein the recombinant gene comprises a nucleic acid sequence of SEQ ID NO:2, SEQ ID NO:3 and/or SEQ ID NO:4 or homologues thereof.

7. The recombinant host of claim 1, wherein the recombinant gene encodes an amino acid sequence of SEQ ID NO: 1 or homologues thereof.

8-17. (canceled)

18. A recombinant host comprising a prototroph comprising a recombinant gene encoding ferritin or a homologue thereof.

19. The recombinant host of claim 18, wherein the recombinant host is a diploid, a polyploid or aneuploid.

20-21. (canceled)

22. The recombinant host of claim 18, wherein the recombinant gene encoding ferritin is chromosomally integrated.

23. The recombinant host of claim 1, wherein the recombinant gene encoding ferritin or a homologue thereof is closely linked to a URA3 gene or a homologue thereof and/or LEU2 or a homologue thereof.

24. The recombinant host of claim 1, wherein the recombinant gene encodes an amino acid sequence for H-ferritin.

25-27. (canceled)

28. The recombinant host of claim 1, wherein the homologue has at least 80% sequence identity with human H-ferritin.

29. The recombinant host of claim 1, wherein the recombinant gene comprises a plant H-ferritin gene.

30. (canceled)

31. A composition comprising the recombinant host of claim 1.

32. The composition of claim 31, wherein the composition further comprises iron.

33-35. (canceled)

36. The composition of claim 32, wherein at least a portion of the iron is complexed with the ferritin or the homologue thereof.

37. (canceled)

38. The composition of claim 31, wherein the recombinant host comprises a bacterium, a virus, a fungus, or combinations thereof.

39-42. (canceled)

43. The composition of claim 31, further comprising a second recombinant host, a probiotic, a prebiotic, or combinations thereof.

44-47. (canceled)

48. An ingestible item comprising the composition of claim 31.

49-51. (canceled)

52. A dietary supplement comprising the composition of claim 31.

53. A pharmaceutical composition comprising the composition of claim 31.

54. A method comprising administering to a subject the composition of claim 31.

55. The method of claim 54, wherein the composition comprises at least 13 mg of iron.

56-60. (canceled)

61. The recombinant host of claim 1, wherein the recombinant gene encoding ferritin is chromosomally integrated.
